(19) United States
(12) Patent Application Publication
(10) Pub. No.: US 2009/0111788 A1
(43) Pub. Date: Apr. 30, 2009

(54) ANTIBIOTIC COMPOSITIONS OF MODIFIED RELEASE AND PROCESS OF PRODUCTION THEREOF

(76) Inventors: Rajesh Jain, New Delhi (IN); Kour Chand Jindal, New Delhi (IN); Munish Talwar, New Delhi (IN)

Correspondence Address: LADAS & PARRY LLP, 26 WEST 61ST STREET, NEW YORK, NY, 10023, US

(21) Appl. No.: 12/294,353

(22) PCT Filed: Mar. 7, 2007
(86) PCT No.: PCT/IN2007/000086

§ 371 Date: Sep. 24, 2008

(30) Foreign Application Priority Data
Mar. 24, 2006 (IN) /DEL/2006

(51) Int. Cl. A61K 31/43; A61K 31/431; A61K 31/545; A61K 31/426; A61K 31/5383; A61P 11/00; A61P 31/04

(52) U.S. Cl. 514/196; 514/192; 514/200; 514/368; 514/230.2

(57) ABSTRACT

Novel modified release pharmaceutical compositions wherein the composition comprises at least one antibiotic(s) preferably amoxicillin or its pharmaceutically acceptable salts, esters, polymorphs, isomers, prodrugs, solvates, hydrates, or derivatives thereof either alone or in combination with other antibiotic(s) as active ingredient, with at least one release modifying agent(s) for controlling the release of the beta lactam antibiotic optionally with one or more other pharmaceutically acceptable excipient(s) is provided, wherein the dosage form provides a release of not more than about 60% of the antibiotic in about 30 minutes and not less than about 70% of the antibiotic after 8 hours when subjected to in vitro dissolution study or when tested in vivo. Further, the compositions of the present invention which when tested in a group of healthy humans provide a mean peak plasma concentration (Cₘₐₓ) after at least about 0.5 hour of administration of the dosage form. The present invention also provides process of preparing such dosage form and methods of using such dosage form.

### FIELD OF INVENTION

The present invention relates to novel modified release pharmaceutical compositions wherein the composition comprises at least one antibiotic(s) preferably amoxicillin or its pharmaceutically acceptable salts, esters, polymorphs, isomers, prodrugs, solvates, hydrates, or derivatives thereof either alone or in combination with other antibiotic(s) as active ingredient, with at least one release modifying agent(s) for controlling the release of the beta lactam antibiotic optionally with one or more other pharmaceutically acceptable excipient(s), wherein the dosage form provides a release of not more than about 60% of the antibiotic in about 30 minutes and not less than about 70% of the antibiotic after 8 hours when subjected to in vitro dissolution study or when tested in vivo. Further, the compositions of the present invention which when tested in a group of healthy humans provide a mean peak plasma concentration (Cₘₐₓ) after at least about 0.5 hour of administration of the dosage form. The present invention also provides process of preparing such dosage form and methods of using such dosage form. The modified release compositions of the present invention, preferably designed for once-a-day or twice-a-day administration, releases the antibiotic(s) in a desired manner so as to maintain therapeutic levels of the active ingredient(s) in vivo for extended periods of time devoid of or at least minimized adverse effects associated with antibiotic therapy, and can be prepared in an easy and cost-effective manner.

### BACKGROUND OF INVENTION

Antibiotics are drugs such as penicillin, streptomycin, and erythromycin that are administered orally or by injection to rid the body of harmful bacteria that cause disease. Several antibiotics are known in literature which belong to different chemical classes and are useful in treating a specific type or various types of bacterial infections depending on the spectrum of activity of the antibiotic. This enormous array of life-saving drugs can be classified into groups based on their chemistry. Included in the penicillin group are penicillin G, the most commonly used penicillin, ampicillin and amoxicillin. Penicillins are used to treat particularly pneumonia, meningitis, streptococcal infections, and sexually transmitted diseases. The cephalosporins, such as cephalothin and cephalexin, share many of their uses with penicillin. The aminoglycosides group includes streptomycin, used chiefly for gram-negative bacterial infections like tuberculosis, and neomycin, which at one time was used to fight systemic infections and has now been replaced in many instances by kanamycin and gentamicin. The tetracylines, including tetracycline and chlortetracycline are broad-spectrum antibiotics that often cause side effects and thus are used in fewer cases. The macrolides include erythromycin, a drug that fights gram-positive bacteria, and is often administered to patients that are allergic to penicillin. Bacitracin belongs to the polypeptide group that is generally effective against gram-negative bacteria. Sulfonamide drugs, such as sulfadiazine, are synthetic drugs used primarily in urinary tract infections often in conjunction with penicillin.

Amoxicillin is a well known beta-lactam antibiotic which has been available for many years. Despite the susceptibility of amoxicillin to inhibition by beta-lactamases produced by resistant microorganisms, amoxicillin still enjoys widespread usage as a broad spectrum antibiotic for the treatment of commonly occurring bacterial infections. In particular, amoxicillin is particularly effective in treating sore throats—acute bacterial tonsillitis and/or pharyngitis where the causative organism is almost exclusively Streptococcus pyogenes. Amoxicillin is available commercially in a variety of formulations, for instance as capsules containing either 250 or 500 mg amoxicillin, as tablets comprising 500 or 875 mg amoxicillin, as chewable tablets comprising either 125 or 250 mg amoxicillin and as dry powder formulation, for reconstitution into an oral suspension. Other formulation types include dispersible tablets providing 500 mg amoxicillin, chewable effervescent tablets, comprising 125, 250 or 500 mg amoxicillin and single dose sachets comprising 750 or 3000 mg amoxicillin. The standard adult dosage is 250 mg three times daily (tid), increasing to 500 mg tid for more severe infections. In addition, the 875 mg tablet is intended for dosing twice daily (bid), as an alternative to the dosage regimen of 500 mg tid. Recently, a 1000 mg chewing tablet has been advertised as being under development (AC Pharma, see SCRIP No 2472, Sep. 15, 1999, page 11). A high dosage of 3 g, bid, is recommended in appropriate cases for the treatment of severe or recurrent purulent infection of the respiratory tract. For short course therapy, in simple urinary tract infections, two 3 g doses, at an interval of 10-12 hours, are given while for a dental abscess; the dosage is two 3 g doses at an interval of 8 h and for gonorrhoea, a single dose of 3 g. Furthermore, the use of 1 g of amoxicillin, bid, is used as one arm of a combination therapy, for eradication of Helicobacter pylori in peptic ulcer disease. Clavulanate is a beta-lactamase inhibitor and is preferably included with the beta-lactam antibiotic amoxicillin to counter a beta-lactamase mediated resistance mechanism. Some microrganisms such as Streptococcus pneumoniae have resistance mechanisms which are not beta-lactamase mediated. PCT Publication No. WO94/16696 discloses generally that potassium clavulanate may enhance the effectiveness of beta-lactam antibiotics such as amoxicillin against microorganisms having a resistance mechanism other than beta-lactamase enzyme mediated resistance. Amoxicillin is provided in combination with the beta-lactamase inhibitor potassium clavulanate, in various tablet formulations of amoxicillin and potassium clavulanate comprising various different weights and ratios of amoxicillin and potassium clavulanate, for instance, conventional swallow tablets comprising 250/125, 500/125, 500/62.5, and 875/125 mg amoxicillin/clavulanic acid (in the form of potassium clavulanate). Such tablets comprise amoxicillin and clavulanic acid in the ratio 2:1, 4:1, 8:1 and 7:1, respectively.

Drug levels can be maintained above the lower level of the therapeutic plasma concentration for longer periods of time by administering larger doses of conventionally formulated dosage forms, but this approach might produce toxic effects due to high plasma concentration of the drug. Alternatively, another approach is to administer a drug at certain intervals of time, resulting in fluctuating drug levels, the so-called peak and valley effect. This approach is generally associated with several potential problems, such as a large peak (toxic effect) and valley (non-active drug level) effect, and a lack of patient compliance leading to drug therapy inefficiency or failure. To overcome such issues, modified release compositions can be formulated with the objective of either releasing the drug in a sustained or controlled manner for an extended period of time or releasing a portion of the drug immediately followed by a sustained or controlled release of drug.

U.S. Pat. No. 6,878,386 discloses a method of treating a bacterial infection in a human in need thereof, which method comprises administering to said human, at a dosage regimen interval of about 12 hours, a dosage of about 2000 mg of amoxicillin and about 125 mg potassium clavulanate, wherein the dosage is delivered from a modified release formulation which has an in vitro dissolution profile wherein about 45% to about 65% of the amoxicillin content is dissolved within 30 min, measured in dissolution test, Apparatus 2, USP 23, 1995, at 37±0.5° C., using deionised water (900 mL) and a paddle speed of 75 rpm. U.S. Pat. No. 6,660,299 discloses modified release pharmaceutical formulation comprising about 2000 mg of amoxicillin in an immediate release phase and a slow release phase; the immediate release phase comprising a first part of amoxicillin formulated with one or more pharmaceutically acceptable excipients which allows for immediate release of the first part of amoxicillin, and the slow release phase comprising a second part of amoxicillin formulated with one or more release modifying pharmaceutically acceptable excipients, wherein the ratio of amoxicillin in the immediate and slow release phase is from 3:1 to 1:3, such that the formulation has an in vitro dissolution profile wherein 45 to 65% of the amoxicillin content is dissolved within 30 min, measured in dissolution test, Apparatus 2, USP 23, 1995, at 37±0.5° C., using 900 mL of deionised water and a paddle speed of 75 rpm. However, such high dosages of amoxicillin disclosed in U.S. Pat. Nos. 6,878,386 and 6,660,299 lead to increase in associated side effects and hence not advisable. U.S. Pat. No. 6,746,692 and US Publication No. 20040241227 relates to modified release formulation of amoxicillin that has an in vitro dissolution profile in which 45% to 65%, preferably 45% to 55% of the amoxicillin content is dissolved within 30 min; further in which 50% to 75%, preferably 55% to 65% of the amoxicillin content is dissolved within 60 min; further in which 55% to 85%, preferably 60% to 70% of the amoxicillin content is dissolved within 120 min; further in which 70% to 95%, preferably 75% to 85% of the amoxicillin content is dissolved within 180 min; and further in which 70% to 100%, preferably 75% to 100% of the amoxicillin content is dissolved within 240 min. In comparison, a conventional, immediate release amoxicillin tablet dissolves essentially completely within 30 minutes. The dissolution profile is measured in a standard dissolution assay, for instance Dissolution Test, Apparatus 2, provided in USP 23, 1995, at 37±0.5° C., using deionised water (900 mL) and a paddle speed of 75 rpm. U.S. Pat. No. 6,756,057 discloses a pharmaceutical formulation of amoxicillin and potassium clavulanate comprising a composition in a solid form of from about 50 to 75 mg bf potassium clavulanate and from about 850 to 1250 mg of amoxicillin; or from about 100 to 150 mg of potassium clavulanate and from about 1700 to 2500 mg of amoxicillin wherein all of the potassium clavulanate and from 0 to 60% of the amoxicillin is in a first release phase and from 40 to 100% of the amoxicillin is in a second release phase; which upon administration to a human provides a T>MIC of at least 4 hours for an MIC of 8 μg/ml.

In addition, the PCT Publication No. WO 97/09042 (SmithKline Beecham) describes tablet formulations comprising amoxicillin and clavulanic acid in a ratio in the range 12:1 to 20:1, preferably 14:1. Furthermore, it is suggested that the preferred dosage of 1750/125 mg may be provided as two tablets, the first comprising 875/125 mg amoxicillin and clavulanic acid and the second 875 mg amoxicillin. The 14:1 ratio is said to be useful for the empiric treatment of bacterial infection potentially caused by drug resistant S. pneumoniae (DRSP). Another PCT Publication No. WO 95/20946 (SmithKline Beecham) describes layered tablets comprising amoxicillin and, optionally a combination with potassium clavulanate, having a first layer which is an immediate release layer and a second layer which is a slow release layer. The broadest ratio of amoxicillin to clavulanic acid is 30:1 to 1:1, with a preferred range of 8:1 to 1:1. Examples provided of such bilayered tablets have amoxicillin trihydrate in the immediate release layer and amoxicillin plus clavulanate in the slow release layer. Multi-layered tablets are described more generically in PCT Publication No. WO 94/06416 (Jagotec AG). Further bilayered tablets comprising clavulanic acid and amoxicillin are described in PCT Publication No. WO 98/05305 (Quadrant Holdings Ltd). In such tablets, a first layer comprises amoxicillin and a second layer comprises clavulanate and the excipient trehalose to stabilise the clavulanate component. Further, the PCT Publication No. WO 95/28148 (SmithKline Beecham) describes amoxicillin/ potassium clavulanate tablet formulations having a core containing amoxicillin and potassium clavulanate coated with a release retarding agent and surrounded by an outer casing layer of amoxicillin and potassium clavulanate. The release retarding agent is an enteric coating, so that there is an immediate release of the contents of the outer core, followed by a second phase from the core which is delayed until the core reaches the intestine. Furthermore, the PCT Publication No. WO 96/04908 (SmithKline Beecham) describes amoxicillin/potassium clavulanate compositions comprising amoxicillin and potassium clavulanate in a matrix, for immediate release, and granules in a delayed release form comprising amoxicillin and potassium clavulanate. Such granules are coated with an enteric coating, so release is delayed until the granules reach the intestine.

Controlled release formulations comprising amoxicillin have been described by several groups. Thus, Arancibia et al (Int J of Clin Pharm, Ther and Tox, 1987, 25, 97-100) describe the pharmacokinetic properties and bioavailability of a controlled release formulation comprising 500 mg of amoxicillin. The formulation was however -designed to release 21% to 35% during the first 60 minutes, 51% to 66% at 4 hours, 70% to 80% at 6 hours, 81% to 90% at 8 hours and more than 94% at 12 hours. They however found little, if any, correlation between the in vitro dissolution rate and the pharmacokinetic behaviour in the body. Hilton et al (International Journal of Pharmaceutics, 1992, 86, 79-88) described an alternative controlled release tablet having a hydrophilic polymer matrix and a gas release system, to provide intragastric buoyancy, to enhance gastric retention time. This showed no advantage over a conventional capsule formulation, with bioavailability being diminished. In contrast, Hilton et al (Journal of Pharmaceutical Sciences, 1993, 82, 737-743) described a 750 mg controlled release tablet incorporating the enteric polymer hydroxypropylmethyl cellulose acetate succinate. This however failed to show any advantage over a conventional capsule. In particular, the bioavailability was reduced to 64.6% compared with the same dosage provided in a capsule. More recently, Hoffman et al (Journal of Controlled Release, 1998, 54, 29-37 and WO 98/22091) have described a tablet comprising 500 mg of amoxicillin in a matrix comprising hydroxypropyl methyl cellulose, designed to release 50% of its contents in the first three hours and complete the drug release process over eight hours. The time above MIC was found to be significantly extended, compared to a capsule formulation, but not enough for a 12 h dosing interval. The discussion is in the context of a theoretical MIC of 0.2 mu.g/ml.

The review of the prior arts therefore suggests that there is still a need to develop novel antibiotic compositions particularly comprising amoxicillin optionally with clavulanate which are safe and highly effective at conventional doses or even at lower doses preferably against more resistant bacteria, and exhibit reduced associated side effects thus providing greater patient compliance. The inventors of the present invention have done extensive research and conducted several experiments to alleviate the drawbacks existing in present art to develop novel modified release antibiotic dosage form compositions particularly comprising amoxicillin optionally with clavulanate by using different excipients to achieve a particular in vitro and in vivo release profile thus demonstrating a significant advancement over the prior art.

### SUMMARY OF THE INVENTION

It is an objective of the present invention to provide modified release pharmaceutical dosage form composition which comprises at least one antibiotic(s) or its pharmaceutically acceptable salts, esters, polymorphs, isomers, prodrugs, solvates, hydrates, or derivatives thereof as active ingredient treated with at least one release modifying agent(s) optionally with one or more other pharmaceutically acceptable excipient(s), wherein the dosage form provides a release of not more than about 60% of the antibiotic in about 30 minutes and not less than about 70% of the antibiotic after 8 hours when subjected to in vitro dissolution study or when tested in vivo.

It is an objective of the present invention to provide modified release pharmaceutical composition which comprises at least one beta-lactam antibiotic(s) preferably amoxicillin or its pharmaceutically acceptable salts, esters, polymorphs, isomers, prodrugs, solvates, hydrates, or derivatives thereof as active ingredient treated with at least one release modifying agent(s) optionally with one or more other pharmaceutically acceptable excipient(s), wherein the dosage form provides a release of not more than about 60% of the beta-lactam antibiotic in 30 minutes and not less than about 70% of the beta-lactam antibiotic after 8 hours when subjected to in vitro dissolution study or when tested in vivo.

It is also an objective of the present invention to provide modified release pharmaceutical composition comprising at least one antibiotic(s), preferably a beta-lactam antibiotic(s), more preferably amoxicillin or its pharmaceutically acceptable salts, esters, polymorphs, isomers, prodrugs, solvates, hydrates, or derivatives thereof as an active ingredient treated with at least one release modifying agent(s) wherein the dosage form composition provides an in vitro release of not more than about 60% of beta-lactam antibiotic in 30 minutes and not less than about 70% of the beta-lactam antibiotic after 8 hours when tested by the USP Apparatus Type II at 75 rpm, 37±0.5° C. and using 900 ml of Distilled water as dissolution media, or equivalent conditions.

It is also an objective of the present invention to provide modified release pharmaceutical composition comprising at least one antibiotic(s), preferably a beta-lactam antibiotic(s), more preferably amoxicillin or its pharmaceutically acceptable salts, esters, polymorphs, isomers, prodrugs, solvates, hydrates, or derivatives thereof as an active ingredient treated with at least one release modifying agent wherein the dosage form composition provides a in vitro release of not more than about 60% of the beta-lactam antibiotic in about 30 minutes and not less than about 70% of the beta-lactam antibiotic after about 8 hours as tested by the USP Apparatus Type II at 75 rpm, 37±0.5° C. and using 900 ml of Distilled water or 0.01N HCl as dissolution media, and when tested in a group of healthy humans (in vivo) the mean peak plasma concentration (Cₘₐₓ) is achieved after at least about 0.5 hour of administration of the dosage form, preferably within 0.5-12 hours.

It is also an objective of the present invention to provide modified release pharmaceutical composition which provides a release of not less than about 80% of the antibiotic after about 8 hours of dissolution study conducted using 900 ml of pH 7.4 Phosphate buffer in USP Apparatus Type II (paddles method) at 75 rpm.

It is also an objective of the present invention to provide modified release pharmaceutical composition which provides a release of about 0-50% of the active ingredient(s) within about 2 hours and greater than about 40% of the active ingredient(s) after about 8 hours of test when subjected to in vitro dissolution study in dissolution media having a pH ranging from about 1 to about 5.5, preferably having a pH of about 1 to about 5.

It is also an objective of the present invention to provide modified release composition comprising amoxicillin trihydrate equivalent to about 300 to about 1900 mg of amoxicillin preferably about 425 mg to about 1500 mg of amoxicillin, and clavulanate potassium equivalent to about 62.5 to about 300 mg of clavulanic acid, preferably about 125 mg to about 250 mg of clavulanic acid with at least one release modifying agent(s) optionally with one or more other pharmaceutically acceptable excipient(s).

It is also an objective of the present invention to provide modified release composition comprising an antibiotic as an active ingredient in combination with at least one other antibiotic.

It is yet another objective of the present invention to provide process of preparation of the composition which comprises treating the antibiotic(s) preferably beta-lactam antibiotic or its pharmaceutically acceptable salts, esters, polymorphs, isomers, prodrugs, solvates, hydrates, or derivatives thereof, with at least one release modifying agent(s) optionally with one or more other pharmaceutically acceptable excipient(s) and formulating it into the desired dosage form.

It is a further objective of the present invention to provide a method of using such novel compositions which comprises administering to a subject in need thereof an effective amount of the composition.

It is also an objective of the present invention to provide method of using of the composition for the management such as prophylaxis, amelioration and/or treatment of bacterial infections which comprises administrating such amount of the composition to a subject in need thereof which provides an effective amount of the antibiotic(s) preferably beta-lactam antibiotic more preferably amoxicillin or its pharmaceutically acceptable salts, esters, polymorphs, isomers, prodrugs, solvates, hydrates, or derivatives thereof, for an extended period of time.

The modified release pharmaceutical compositions of the present invention preferably designed for once-a-day or twice-a-day administration releases the antibiotic(s) in a desired manner particularly in vivo so as to maintain therapeutic levels of the drug for extended periods of time devoid of or at least minimized adverse effects associated with antibiotic therapy, and can be prepared in an easy and cost-effective manner.

### DETAILED DESCRIPTION OF THE INVENTION

It is an objective of the present invention to provide modified release pharmaceutical dosage form composition which comprises at least one antibiotic(s) or its pharmaceutically acceptable salts, esters, polymorphs, isomers, prodrugs, solvates, hydrates, or derivatives thereof as active ingredient treated with at least one release modifying agent(s) optionally with one or more other pharmaceutically acceptable excipient(s), wherein the dosage form provides a release of not more than about 60% of the antibiotic in about 30 minutes and not less than about 70% of the antibiotic after about 8 hours when subjected to in vitro dissolution study or when tested in vivo. Preferably the active ingredient is a beta-lactam antibiotic(s), more preferably amoxicillin or its pharmaceutically acceptable salts, esters, polymorphs, isomers, prodrugs, solvates, hydrates, or derivatives thereof. The release profile as stated herein refers to either in vitro release profile of the antibiotic(s) as obtained by dissolution study or in vivo release profile of the antibiotic(s) tested in particularly humans, or both. In an embodiment, the release modifying agent(s) is preferably a mucoadhesive polymer.

In an embodiment, the present invention provides modified release pharmaceutical composition comprising at least one antibiotic(s), preferably a beta-lactam antibiotic(s), more preferably amoxicillin or its pharmaceutically acceptable salts, esters, polymorphs, isomers, prodrugs, solvates, hydrates, or derivatives thereof as an active ingredient treated with at least one release modifying agent(s) wherein the dosage form composition provides a release of not more than about 60% of the beta-lactam antibiotic in about 30 minutes and not less than about 70% of the beta-lactam antibiotic after about 8 hours when tested by the USP Apparatus Type II at 75 rpm, 37±0.5° C. and using 900 ml of Distilled water (referred to herein as ‘Media-I’) or 0.01N HCl as dissolution media (referred to herein as ‘Media-II’).

In yet another embodiment, the modified release pharmaceutical composition of the present invention exhibits a release profile in the pH 7.4 Phosphate buffer dissolution media using USP Apparatus Type II (paddles method) at 75 rpm (referred to herein as ‘Media-III’), which comprises releasing not less than about 80% of the antibiotic after about 8 hours of study.

In a further embodiment, the compositions of the present invention comprising pharmaceutically active agent(s) were subjected to in vitro dissolution study in dissolution media having a pH ranging from about 1 to about 5.5, preferably having a pH of about 1 to about 5 using USP Apparatus Type II (paddles method). About 0-50% of the active ingredient(s) was released within about 2 hours and greater than about 40% of the active ingredient(s) was released after 8 hours of test. However, it might be emphasized that the selection of the in vitro dissolution study media, the parameters and apparatus is made in such a manner so as to provide a scientific rationale to the intended study and/or a logical correlation to the in vivo data as understood by a person skilled in art, and any modifications in such study either in vitro or in vivo is within the purview of the present invention.

In an embodiment of the present invention, the pharmaceutical dosage form composition comprises a plurality of particles, wherein each particle comprises at least one antibiotic(s) or its pharmaceutically acceptable salts, esters, polymorphs, isomers, prodrugs, solvates, hydrates, or derivatives thereof, treated with at least one release modifying agent(s) optionally with one or more pharmaceutically acceptable excipient(s) for controlling the release of the antibiotic(s).

In an embodiment, the active ingredient of the present invention is selected from but not limited to a group comprising antibiotics, preferably beta-lactam antibiotics such as cephalosporins and penicillins, for example, amoxicillin, ampicillin, bacampicillin, carbenicillin, cloxacillin, dicloxacillin, flucloxacillin, methicillin, mezlocillin, nafcillin, oxacillin, penicillin G, penicillin V, piperacillin, pivampicillin, pivmecillinam, ticarcillin, clavulanic acid; or other antibiotics such as ciprofloxacin, ofloxacin, levofloxacin, and the like or mixtures thereof, or pharmaceutically acceptable salts, esters, polymorphs, isomers, prodrugs, solvates, hydrates, or derivatives thereof. In a further embodiment, the dosage form of the present invention comprises at least two antibiotics as active ingredients.

In an embodiment, the dosage form comprises amoxicillin as the active ingredient in at least about 20% preferably at least about 50% by weight of the dosage form. In another embodiment, the modified release dosage form of the present invention is in the extended release form, sustained release form, timed release form, pulsatile release form, prolonged release form or delayed release form, or in a combination of immediate release form and extended release form. In a preferred embodiment, one antibiotic active ingredient in the modified release dosage form is amoxicillin or a pharmaceutically acceptable salt, ester, solvate, polymorphs, isomers, prodrug, or derivative thereof present in an extended release form, whereas the other antibiotic is present in an immediate release form. In another embodiment, at least one part of an antibiotic, preferably amoxicillin is present in an extended release form, whereas at least another part of an antibiotic, preferably amoxicillin is present in an immediate release form. Preferably the modified release composition of the present invention comprises amoxicillin trihydrate in extended release form and clavulanate potassium in an immediate release form. In another embodiment, the modified release composition comprises amoxicillin trihydrate equivalent to about 300 to about 1900 mg of amoxicillin preferably about 425 mg to about 1500 mg of amoxicillin, and clavulanate potassium equivalent to about 62.5 to about 300 mg of clavulanic acid, preferably about 125 mg to about 250 mg of clavulanic acid with at least one release modifying agent(s) optionally with one or more other pharmaceutically acceptable excipient(s).

In an embodiment, the modified release pharmaceutical dosage form composition comprises amoxicillin formulated with at least one release modifying agent(s) and one or more other pharmaceutically acceptable excipient(s) to provide an extended release of amoxicillin, and potassium clavulanate in an immediate release form to provide immediate or fast release of clavulanate. In an embodiment, the potassium clavulanate provides a release of not less than about 20% of the antibiotic in about 2 hours and about 75% in about 1 to about 15 hours when subjected to in vitro test using USP Apparatus Type II at 75 rpm, 37±0.5° C. and using 900 ml of Distilled water (referred to herein as ‘Media-I’) or 0.01N HCl as dissolution media (referred to herein as ‘Media-II’).

In an embodiment, the novel modified release pharmaceutical compositions of the present invention is intended to reduce the adverse effects or side effects associated with the antibiotic(s) by controlling the peak plasma concentration (Cₘₐₓ) such that the concentration of the antibiotic(s) are substantially below their toxic levels at any point of time although the plasma concentration of the antibiotic(s) is above the MIC (minimum inhibitory concentration) for such period adequate to provide the therapeutic efficacy. Also the steady state concentrations of the antibiotic(s) do not exhibit substantial fluctuations. The reduced incidence of the side effects is thus intended to improve patient compliance with the therapy. In another embodiment of the present invention, the inventors have surprisingly found the role of the pharmaceutical excipient(s) preferably the release controlling agent in reducing the side effects particularly in the form of gastrointestinal disorders/disturbances related to the antibiotic(s) therapy. Particularly it has been found that the use of a mucoadhesive polymer such as polycarbophil or polyethylene oxide has an effect in reducing the gastrointestinal disorders which arises primarily due to the destruction of the useful microbial flora of the GIT during the antibiotic therapy and/or the detrimental effect of the antibiotic(s) on the gastrointestinal tract.

For beta-lactams, including amoxicillin, it is recognised that the time above minimum inhibitory concentration (T>MIC) is the pharmacodynamic parameter most closely related to efficacy. For a variety of beta-lactams, a bacteriological cure rate of 85 to 100% is achieved when serum concentrations exceed the MIC for more than about 40% of the dosing interval. In an embodiment of the present invention, the time over MIC (T>MIC) for the antibiotic compositions is at least 40% at a concentration of at least about 0.25 μg/ml of the antibiotic at this MIC. The antibiotic compositions of the present invention provide therapeutic levels of the active ingredient at concentrations of about 0.25 μg/ml of the antibiotic for at least about 4-6 hours after administration or for such time as required to provide effectiveness of the antibiotic.

A further parameter which is of importance for effective antibiotic therapy is the ratio of the maximum plasma concentration (Cₘₐₓ) to the MIC value, as this may be related to the potential for resistance. Too low a ratio may encourage the development of resistant strains. In an embodiment, the compositions of the present invention preferably have such a Cₘₐₓ to the MIC ratio so as to avoid or at least minimize development of resistant microbial strains. In a further embodiment, the compositions of the present invention preferably have a Cₘₐₓ value which is well above MIC value, for instance, at least two times or at least three times the MIC value.

The compositions of the present invention are prepared by using formulation techniques aimed at modified release of the beta-lactam antibiotic in a manner such that the bioavailability of dosage form thus obtained is at least comparable to a conventional immediate release dosage form preferably administered in the fed state and also shows lesser degree of adverse effects. In an aspect, the release of the beta-lactam antibiotic from the dosage form of the present invention is controlled in a manner by using release modifying agent(s) such that therapeutically effective plasma concentration of the antibiotic can be obtained without any undesirable side effects for an extended period of time thus leading to improved patient compliance.

In another embodiment, the formulation of this invention will normally, in addition to its active ingredient(s) preferably amoxicillin trihydrate and potassium clavulanate, also include excipients which are standard in the field of formulations for oral dosing and used in generally standard proportions, and at generally standard particle sizes and grades, etc. In the case of oral suspensions, these excipients may comprise suspending aids, glidants (to aid filling), diluents, bulking agent, flavours, sweeteners, stabilisers, and in the case of dry formulations for make up to an aqueous suspension, an edible desiccant to assist preservation of the potassium clavulanate against hydrolysis by atmospheric moisture on storage. Potassium clavulanate is normally supplied in admixture with microcrystalline cellulose or silicon dioxide as diluent.

In an embodiment of the present invention, the release modifying agent used in the dosage form is selected from but not limited to a group comprising carbopol; cellulosic polymers such as sodium carboxymethyl cellulose, hydroxypropyl cellulose, hydroxypropylmethyl cellulose, hydroxyethyl cellulose, methyl cellulose; copolymers of methyl vinyl ether and maleic anhydride such as Gantrez®; enteric polymers; sodium hyaluronate; gums; alginates; polycarbophil; polyethylene oxide; starch; dextran; chitosan; and the like or mixtures thereof.

In a further embodiment, the release modifying agent of the present invention comprises a polymeric material selected from but not limited to the group comprising pH dependent polymers; pH independent polymers; swellable polymers; non-swellable polymers; hydrophilic polymers; hydrophobic polymers and/or one or more other hydrophobic materials; ionic polymers such as sodium alginate, carbomer, calcium carboxymethylcellulose or sodium carboxymethylcellulose; non-ionic polymers such as hydroxypropyl methylcellulose; synthetic or natural polysaccharide selected from the group comprising alkylcelluloses, hydroxyalkyl celluloses, cellulose ethers, cellulose esters, nitrocelluloses, dextrin, agar, carrageenan, pectin, furcellaran, starch and starch derivative, and mixtures thereof. The polymeric material used in the present invention is selected from but not limited to a group comprising cellulosic polymer, methacrylate polymer, methacrylate copolymer such as Eudragit® EPO, Eudragit® E100, Eudragit® E12,5 and the like or mixtures thereof, Polyvinylpyrollidone (PVP), alginate, polyvinylpyrrolidone-polyvinyl acetate (PVP-PVA) copolymer, ethylcellulose, cellulose acetate, cellulose propionate (lower, medium or higher molecular weight), cellulose acetate propionate, cellulose acetate butyrate, cellulose acetate phthalate, cellulose triacetate, poly(alkyl methacrylate), poly(isodecyl methacrylate), poly(lauryl methacrylate), poly(phenyl methacrylate), poly(alkyl acrylate), poly(octadecyl acrylate), poly(ethylene), poly(alkylene), poly(alkylene oxide), poly(alkylene terephthalate), poly(vinyl isobutyl ether), poly(vinyl acetate), poly(vinyl chloride) and polyurethane or a mixture thereof used either alone or in combination thereof. In a further embodiment, the dosage form additionally comprises a gum selected from but not limited to a group comprising xanthan gum, guar gum, gum arabic, carrageenan gum, karaya gum, locust bean gum, acacia gum, tragacanth gum, agar and the like or mixtures thereof.

In another embodiment, the dosage form of the present invention additionally comprises at least one surfactant selected from a group comprising anionic surfactants, cationic surfactants, non-ionic surfactants, zwitterionic surfactants or mixtures thereof. Other pharmaceutically acceptable excipients used in the composition of the present invention are selected from but not limited to a group of excipients generally known to persons skilled in the art e.g. diluents such as lactose, mannitol, sorbitol, starch, microcrystalline cellulose, xylitol, fructose, sucrose, dextrose, dicalcium phosphate, calcium sulphate; disintegrants; binders; fillers; bulking agent; organic acid(s); colorants; stabilizers; preservatives; lubricants; glidants; chelating agents; vehicles; bulking agents; stabilizers; preservatives; hydrophilic polymers; solubility enhancing agents such as glycerine, various grades of polyethylene oxides, transcutol and glycofurol; tonicity adjusting agents; local anesthetics; pH adjusting agents; antioxidants; osmotic agents; chelating agents; viscosifying agents; acids; sugar alcohol; reducing sugars; non-reducing sugars and the like used either alone or in combination thereof. The disintegrants used in the present invention include but not limited to a group comprising croscarmellose sodium (e.g. Primellose®), sodium starch glycollate, cross-linked sodium carboxymethyl cellulose (e.g. Ac-di-sol®), Solutab®, Vivasol®, starches, pregelatinized starch, celluloses, cross-linked carboxymethylcellulose, crospovidone, clays, alginates, gums and the like used either alone or in combination thereof. The diluents or fillers useful in the present invention are selected from but not limited to a group comprising lactose, starch, mannitol, sorbitol, dextrose, microcrystalline cellulose, dibasic calcium phosphate, sucrose-based diluents, confectioner's sugar, monobasic calcium sulfate monohydrate, calcium sulfate, calcium lactatc, dextrose, dextran, dextrates, inositol, hydrolyzed cercal solids, amylose, powdered cellulose, calcium carbonate, cellulose powder, starches, pregelatinized starch, sucrose, xylitol, lactitol, mannitol, sorbitol, sodium chloride, polyethylene glycol, glycine, or bentonites, and the like. The lubricants used in the present invention are selected from but not limited to a group comprising talc, magnesium stearate, calcium stearate, zinc stearate, stearic acid, hydrogenated vegetable oil, sodium stearyl fumarate, glyceryl behenate, waxes and the like used either alone or in combination thereof. The anti-adherents or glidants are selected from but not limited to a group comprising talc, corn starch, DL-leucine, sodium lauryl sulfate, magnesium stearate, calcium stearate, sodium stearate, colloidal silicon dioxide, and the like. The vehicles suitable for use in the present invention can be selected from but not limited to a group comprising dimethylacetamide, dimethylformamide and dimethylsulphoxide of N-methyl pyrrolidone, benzyl benzoate, benzyl alcohol, ethyl oleate, polyoxyethylene glycolated castor oils (Cremophor® EL), polyethylene glycol MW 200 to 6000, propylene glycol, hexylene glycols, butylene glycols and glycol derivatives such as polyethylene glycol 660 hydroxy stearate (commercially available as Solutrol® HS15). In another embodiment of the present invention, the compositions may additionally comprise an antimicrobial preservative such as Benzyl alcohol preferably at a concentration of 2.0% v/v of the composition. In an embodiment of the present invention, the composition may additionally comprise a conventionally known antioxidant such as ascorbyl palmirate, butyl hydroxy anisole, butyl hydroxy toluene, propyl gallate, α-tocopherol, and the like or mixtures thereof.

In an embodiment, the compositions of the present invention may additionally comprise of a colorant in order to produce a desirable colour. Any type of colour known to be ‘FD&C’ certified may be used to provide colouring to the product. Suitable colorants include natural colorants, i.e., pigments and dyes obtained from mineral, plant, and animal sources. Examples of natural colorants include red ferric oxide, yellow ferric oxide, annattenes, alizarin, indigo, rutin, quercetin, and the like. Synthetic colorants may also be used, which is typically an FD&C or D&C dye, e.g., an approved dye selected from the so-called ‘coal-tar’ dyes, such as a nitroso dye, a nitro dye, an azo dye, an oxazine, a thiazine, a pyrazolone, a xanthene, an indigoid, an anthraquinone, an acridine, a rosaniline, a phthalein, a quinoline, or a ‘lake’ thereof, i.e., an aluminum or calcium salt thereof. Particularly preferred colorants are food colorants in the ‘GRAS’ (Generally Regarded As Safe) category.

In another embodiment of the present invention, the release modifying agent is a mucoadhesive polymer or combination of such polymers such as polycarbophil and/or polyethylene oxide having mucin binding property which is a key feature of an Enhanced Activity Drug Delivery System (EADDS). The polymer alongwith the active ingredient adheres to the mucosal surface thereby enhancing the availability of the active ingredient at the site of action where the microorganism(s) reside. Further, when the composition of the present invention disintegrates upon in vivo administration into multiparticles/fragments, the mucoadhesion of these multiparticles/fragments preferably limit the site of antibiotic(s) absorption to upper gastric part only, thus preventing the undue exposure of antibiotic(s) to intestinal microorganisms and in turn preventing or at least minimizing the associated side effects like diarrhoea. This in turn helps to increase the patient compliance towards drug therapy and prevents the development of resistance in microorganisms.

In an embodiment of the present invention is provided a process of preparation of the dosage form which comprises treating the antibiotic(s), preferably a beta-lactam antibiotic, more preferably amoxicillin or its pharmaceutically acceptable salts, esters, prodrugs, solvates, hydrates, or derivatives thereof with at least one release modifying agent(s) optionally with other pharmaceutically acceptable excipient(s) and formulating it into the desired dosage form.

The pharmaceutical dosage form composition of the present invention is preferably formulated as an oral dosage form either as a solid, semi-solid, gel, or a liquid preparation such as tablets, capsules, patches, powders, granules, dry syrup, suspension, topical gels, solutions, emulsions, and the like. In an embodiment, the composition of the present invention is preferably a solid oral dosage form, more preferably in the form of tablets. The tablets can be prepared by either direct compression, dry compression (slugging), or by granulation. The granulation technique is either aqueous or non-aqueous. The non-aqueous solvent used is selected from a group comprising ethanol, isopropyl alcohol, methylene chloride, or mixtures thereof. Powder or granular formulations, such as paediatric suspension formulations, may be manufactured using techniques which are generally conventional in the field of manufacture of pharmaceutical formulations and in the manufacture of dry formulations for reconstitution into such suspensions. For example a suitable technique is that of mixing dry powdered or granulated ingredients for loading into a suitable container. In an embodiment, the compositions of the present invention are in the form of compressed tablets, moulded tablets, products prepared by extrusion or film cast technique, and the like. For paediatric dosing, the formulations of the invention are preferably made up into a sweet flavoured aqueous syrup formulation of generally conventional formulation (except for its novel amoxicillin: clavulanate ratio and intended use) containing a suitable weight of the amoxicillin and clavulanate in a unit dose volume, e.g. 5 ml or 2.5 ml of the syrup. Because of the water-sensitivity of clavulanate it is 5 preferred to provide such a syrup formulation as dry powder or granules contained in an atmospheric moisture-proof container or sachet for make up with water or other suitable aqueous medium shortly prior to use.

In another embodiment, the composition of the present invention can be formulated into a dosage form selected from the group consisting of liquid dispersions, oral suspensions, gels, aerosols, ointments, creams, controlled release formulations, fast melt formulations, lyophilized formulations, delayed release formulations, extended release formulations, pulsatile release formulations, and mixed immediate release and controlled release formulations. The compositions of the present invention can be formulated as gastro-retentive dosage forms wherein gastro-retentivity is achieved either making the size of the dosage form such that it is bigger than the size of the gastro-intestinal tract or by making dosage the form which float in the contents of the gastrointestinal tract and thus gets retained or by making the dosage form as a mucoadhesive type wherein the intact dosage form or the plurality of particles arising out of the rapid disintegration of the mucoadhesive dosage form stick to the gastric mucosa and remain for an extended period of time thus providing a controlled release of the active ingredient in vivo. The release of the active ingredient from the compositions of the present invention preferably does not depend on the food intake, thus avoiding the food effect or at least showing a reduction in variability associated with the administration of the dosage form in the fed state. Further, the compositions of the present invention are expected not to compromise the bioavailability of the active ingredient under fed or fasted conditions.

In an embodiment, the present invention provides a method of using such novel compositions which comprises administering to a subject in need thereof an effective amount of the composition. In an embodiment, the present invention provides method of using of the composition for the management such as prophylaxis, amelioration and/or treatment of bacterial infections which comprises administrating such amount of the composition to a subject in need thereof which provides an effective amount of the antibiotic(s) preferably beta-lactam antibiotic more preferably amoxicillin or its pharmaceutically acceptable salts, esters, polymorphs, isomers, prodrugs, solvates, hydrates, or derivatives thereof, for an extended period of time. The compositions are particularly useful for the treatment of common bacterial infections primarily the upper respiratory tract infections such as sore throat, acute bacterial tonsillitis and/or pharyngitis, and the like or a combination of such disorders, especially for treatment of bacterial infections occurring due to more than one microorganisms such as different gram positive or gram negative bacteria.

In an embodiment, the dosage form composition of the present invention provides an in vitro release of not less than about 5% and not more than about 70% of the antibiotic particularly amoxicillin after 0.5 hours; from not less than about 15% amoxicillin is released in 3 hours; and not less than about 60% amoxicillin is released in 6 hours as tested by the USP Apparatus Type II at 75 rpm, 37±0.5° C. using 900 ml of Distilled water as the dissolution medium.

In an embodiment, a dissolution study methods of the present invention have the following parameters:

Dissolution media (900 ml): Distilled water or 0.01N Hydrochloric acid (HCL) or pH 7.4 phosphate buffer

| | |
|---|---|
| Apparatus | USP Apparatus Type II (Paddle) |
| Paddle Speed | 75 rpm |
| Temperature of dissolution medium | 37° C. ± 0.5° C. |

Illustrated herein is an embodiment of the present invention which describes a method to carry out the in-vitro dissolution study of amoxicillin using 900 ml of Distilled water as the dissolution medium. Alternative dissolution methods for amoxicillin or other beta-lactam antibiotics can be used by making the necessary modifications specific to the properties of the active ingredient and the specific drug release (dissolution) medium used in the in vitro study. The active ingredient (drug) release was analyzed and measured by UV-Spectroscopy using a UV/VIS Spectrophotometer. Alternative analytical instruments such as HPLC or any other instrument known to the art can be used for analysis of the active ingredient(s).

Dissolution Procedure: The dissolution apparatus was set by programming the temperature, rotation and run time at 37° C.±0.5° C., 75 rpm and 12 hours respectively. 900 ml of Distilled water (dissolution medium) was placed in each of the six vessels of the dissolution apparatus. The apparatus was assembled and the dissolution medium was equilibrated to 37° C.±0.5° C. and the thermometer was removed. One unit dosage was placed in each of the six vessels. Rotation of the paddle was started immediately at the speed of 75 rpm for 12 hours. Sampling intervals selected were 0.5, 1.0, 2.0, 3.0, 4.0, 6.0, 8.0 and 12.0 hours. Aliquots were withdrawn, and successively replaced with equal volumes of fresh dissolution medium, at the desired interval periods from a zone midway between the surface of the dissolution medium and top of the rotating blades, from each of the six vessels and the step was proceeded as given under ‘Test preparation’. The vessel was covered during the test and the temperature of the medium was verified at specific intervals.

Buffer solution preparation: 6.804 g of potassium dihydrogen phosphate was dissolved in 1000 ml of water. The pH was adjusted to 5.0±0.05 with potassium hydroxide solution.

Standard preparation: About 80.0 mg of Amoxicillin trihydrate WS (Working Standard) was weighed and transferred accurately into a 100 ml volumetric flask. Amoxicillin was dissolved and the volume was made up with water followed by mixing. Filtration through 0.45 μm membrane filter (Millipore HVLP Type) was carried out, discarding first 5 ml of the filtrate. 2.0 ml of the resulting filtrate was diluted to 100 ml with buffer solution followed by mixing.

Test preparation: Each of the dissolution samples withdrawn through 0.45 μm membrane filter (Millipore HVLP Type) was filtered discarding first 5.0 ml of the filtrate. 2.0 ml of the above filtrate was diluted to 100 ml with the buffer solution followed by mixing.

Blank preparation: 2.0 ml of Distilled water was accurately transferred to a 100 ml volumetric flask and diluted to volume with buffer solution followed by mixing.

Procedure: The absorbance of each of the Standard preparation and Test preparations withdrawn at different intervals was measured by UV/VIS spectrophotometer at about 228 nm by using dissolution medium as a blank. The quantity of amoxicillin released in percentage with respect to claimed values in the present Test preparations withdrawn at different intervals was calculated using the below mentioned formulae. For example, for the first sampling point i.e. after 0.5 hour and the last sampling point i.e. after 12.0 hours, the formulas are:

$$After 0.5 hour := \frac{{Ab}_{T}}{{Ab}_{S}} \times \frac{{W}_{S}}{100} \times \frac{2}{100} \times \frac{900}{C} \times \frac{100}{2} \times \frac{P}{100} \times 100 \\ After 12.0 hours := \frac{{Ab}_{T}}{{Ab}_{S}} \times \frac{{W}_{S}}{100} \times \frac{2}{100} \times \frac{900}{C} \times \frac{100}{2} \times \frac{P}{100} \times 100 ) + C R$$

### Where,

Ab_{T}=Absorbance of test preparation.

Ab_{S}=Absorbance of standard preparation.

W_{S}=Weight of Amoxicillin WS taken (in mg).

P=Potency of Amoxicillin WS (in % w/w).

C=Claim value of Amoxicillin in each unit dosage.

CR=Corrected release for Amoxicillin, in %, at different intervals (i.e. at 1, 2, 3, 4, 6, 8 and 12 hours)

Similarly, the quantity of amoxicillin released in percentage with respect to claimed values in the present Test preparations withdrawn at other time intervals such as at 1.0, 2.0, 3.0, 4.0, 6.0 and 8.0 hours are calculated using similar formulas.

The influences of various process parameters on the Dissolution Rate of the beta-lactam antibiotic dosage form composition of the present invention were evaluated. The investigations by the inventors have indicated that the dissolution rate of the beta-lactam antibiotic is dependant on the excipients used in the composition and manufacturing process employed to make the composition.

In a further embodiment, the said dosage form of the present invention, when tested in a group of healthy humans, the mean peak plasma concentration (Cₘₐₓ) is achieved after at least about 0.5 hour of administration of the dosage form, preferably within about 0.5-12 hours, more preferably within about 1-8 hours. In yet another embodiment of the present invention, the compositions when tested in a group of at least twelve healthy humans showed a mean peak plasma concentration (Cₘₐₓ) of amoxicillin in the range of about 0.1-50 μg/ml, preferably in the range of about 3-30 μg/ml.

A comparative bio-availability (in vivo) study of an amoxicillin modified release formulations of the present invention was carried out against Moil® tablets (Galax SmithKline) in a group of healthy human volunteers. The aim of the study was to undergo comparative pharmacokinetic evaluation of four modified release tablet formulations containing namely 375 mg (referred to as ‘T-1’), 425 mg (referred to as ‘T-2’), 625 mg (referred to as ‘T-3’) and 750 mg (referred to as ‘T-4’) of Amoxicillin. The said compositions T-1, T-2, T-3 and T-4 were prepared according to the composition disclosed under example-1 herein. The Amoxicillin modified release tablets (TEST compositions i.e. T-1 & T-2) were evaluated against Amoxicillin 500 mg conventional release tablet (Moil® 500 mg referred to as ‘REFERENCE’ i.e. R-1), and the Amoxicillin modified release tablets (TEST compositions i.e. T-3 & T-4) were evaluated against Amoxicillin 875 mg conventional release tablet (Moil® 875 mg referred to as ‘REFERENCE’ i.e. R-2) in healthy human volunteers, before and after food, using a randomized, open label, balanced, three-treatment, three-period, three-sequence, single-dose cross over design. The study design involved twelve healthy human volunteers aged between 18-45 years, weighing 70.1±8 kegs with a mean BMI (Body Mass Index) of 16.9±1.9. Two studies namely fed and fasted studies were conducted by giving the formulations after heavy breakfast and fasting conditions respectively. After a supervised overnight fast for 12 hours and after consuming whole high-fat breakfast within 30 minutes, study was conducted on volunteers with a single oral dose of a TEST/REFERENCE composition administered with 240 ml of water. Drug analysis was done by collecting blood samples in vials through indwelling canola/clean vein puncture throughout the study at redoes, 0.25, 0.5, 0.75, 1.0, 2.0, 3.0, 4.0, 5.0, 6.0, 7.0, 8.0, 10.0, 12.0, and 14.0 hours after the administration of TEST/REFERENCE compositions. The blood samples were collected in sample collection tubes coated with sodium heparin as the anticoagulant. The heparins plasma obtained was separated from blood by centrifugation and the plasma samples were stored at −20° C. till the last sample was collected and after transferred to −75±5° C. until analysis. The various pharmacokinetic parameters were evaluated namely Cₘₐₓ (peak plasma concentration of drug), Tₘₐₓ (time to reach peak plasma concentration), AUC₀₋ₜ (Area under the ‘plasma concentration versus time’ curve from time=0 to time=t where ‘t’ denotes the time of last measurable concentration), AUC_{0-α}, (Area under the ‘plasma concentration versus time’ curve from time=0 to time=α, where ‘α’ denotes infinity) and t_{1/2} (plasma elimination half life). The statistical and pharmacokinetic analyses were generated using WinNonlin® software (version 5.0). The data of pharmacokinetic parameters is presented in the Table-1 and Table-2 below. The percent Time above MIC for the ‘REFERENCE’ (R-1) and TEST compositions (T-1 & T-2) and similarly for ‘REFERENCE’ (R-1) and TEST compositions (T-1 & T-2) at various plasma concentrations of the drug were also measured. The data is presented in Table-3 and Table-4 respectively.

TABLE 1
Comparative pharmacokinetic parameters of ‘REFERENCE’ (R-1)
and TEST compositions (T-1 &T-2) in the fed state

| pK Parameters | R-1 | T-1 | T-2 |
|---|---|---|---|
| Tₘₐₓ (hrs) | 2.033 | 2.429 | 2.20 |
| Cₘₐₓ (μg/ml) | 6.219 | 4.154 | 4.088 |
| AUC Last (μg/ml/hr) | 16.726 | 14.879 | 14.358 |
| AUC_{0-∞} (μg/ml/hr) | 17.042 | 15.206 | 14.902 |

TABLE 2
Comparative pharmacokinetic parameters of ‘REFERENCE’ (R-2)
and TEST compositions (T-3 &T-4) in the fed state

| pK Parameters | R-2 | T-3 | T-4 |
|---|---|---|---|
| Tₘₐₓ (hrs) | 2.10 | 2.143 | 2.633 |
| Cₘₐₓ (μg/ml) | 10.393 | 5.50 | 6.007 |
| AUC Last (μg/ml/hr) | 27.774 | 18.105 | 22.712 |
| AUC_{0-∞} (μg/ml/hr) | 28.036 | 18.404 | 22.962 |

TABLE 3
Percent Time above MIC for the ‘REFERENCE’ (R-1) and TEST
compositions (T-1 &T-2)

| | | | | Con- |
|---|---|---|---|---|
| | Concentration | Concentration | Concentration | centration |
| Treatment | (0.25 μg/ml) | (1 μg/ml) | (1.6 μg/ml) | (2 μg/ml) |
| R-1 | 60.00 | 39.16 | 32.50 | 28.33 |
| T-2 | 69.16 | 45.83 | 33.33 | 27.50 |
| T-1 | 71.66 | 43.33 | 34.16 | 28.33 |

TABLE 4
Percent Time above MIC for the ‘REFERENCE’ (R-2) and TEST
compositions (T-3 &T-4)

| | Concen- | | Concen- | |
|---|---|---|---|---|
| | tration | Concentration | tration | Concentration |
| Treatment | (0.25 μg/ml) | (1 μg/ml) | (1.6 μg/ml) | (2 μg/ml) |
| R-2 | 70.83 | 47.50 | 40.83 | 38.33 |
| T-4 | 76.66 | 52.50 | 44.16 | 40.00 |
| T-3 | 71.66 | 46.66 | 38.33 | 34.16 |

The study indicated that the TEST compositions, even at lower doses, showed pharmacokinetic parameters and also the ‘Percent Time above MIC’ values at different plasma concentrations which when compared with the REFERENCE product were found to be adequate for obtaining the desired therapeutic response for extended periods of time. The study also showed that the TEST products T-1 and T-2 did not show significant differences in the pharmacokinetic parameters. This study thus proves that the compositions of the present invention showed superior or at least comparative bioavailability of the active ingredient even at significantly lower doses as compared to the REFERENCE product. Hence the compositions of the present invention provide a significant advancement in designing novel dosage forms comprising an antibiotic, which not only has a comparative efficacy even at lower doses but also aids in reducing the dose related adverse events associated with antibiotic therapy, thus providing a better patient compliance. The examples of pharmaceutical compositions given below serve to illustrate embodiments of the present invention. However, they do not intend to limit the scope of present invention.

### EXAMPLES

### Example—1

### A. Preparation Of Granules

| | | Quantity/tablet (mg) | | | |
|---|---|---|---|---|---|
| S. No. | Ingredients | T-1 | T-2 | T-3 | T-4 |
| 1. | Amoxicillin trihydrate* | 431.25 | 488.75 | 718.75 | 862.50 |
| 2. | Polyethylene oxide | 25.00 | 28.33 | 41.67 | 50.00 |
| 3. | Polycarbophil | 10.00 | 11.33 | 16.67 | 20.00 |
| 4. | Lactose | 15.00 | 17.00 | 25.00 | 30.00 |
| 5. | Croscarmellose sodium | 12.50 | 14.17 | 20.83 | 25.00 |
| 6. | Purified water** | q.s. | q.s. | q.s. | q.s. |

Procedure:

i) Amoxicillin trihydrate, Polyethylene oxide, Lactose, Croscarmellose sodium and Polycarbophil were passed through sieve #30 followed by mixing.

ii) The blend of step (i) was granulated with Purified water.

iii) The wet mass of step (ii) was passed through sieve #8.

iv) The granules of step (iii) were semi-dried at a temperature of 50° C. and passed through sieve #24 followed by breaking the lumps retained on the sieve.

v) The granules of step (iv) were passed through sieve #80 and further collected.

vi) The undersize granules obtained in step (v) were milled followed by regranulating the granules with purified water. The process of step (iii) was repeated until at least 95% of the material of +#24 and −#80 fraction was obtained.

vii) The total fraction of granules obtained were blended and stored in double polyethylene bags in tightly closed HDPE containers.

(‘*’ indicates Amoxicillin trihydrate 431.25, 488.75, 718.75 & 862.50 mg is equivalent to 375, 425, 625 & 725 mg of Amoxicillin respectively)

### B. Coating Of Granules

| S. | | Quantity/tablet (mg) | | | |
|---|---|---|---|---|---|
| No. | Ingredients | T-1 | T-2 | T-3 | T-4 |
| 7. | Methacrylic acid copolymer, | 98.50 | 111.63 | 164.17 | 197.00 |
| | Type A (Eudragit ® L-100) | | | | |
| 8. | Polycarbophil | 1.50 | 1.70 | 2.50 | 3.00 |
| 9. | Triethyl citrate | 9.85 | 11.16 | 16.42 | 19.70 |
| 10. | Iron oxide red | 0.63 | 0.71 | 1.04 | 1.25 |
| 11. | Isopropyl alcohol** | q.s. | q.s. | q.s. | q.s. |
| 12. | Purified water** | q.s. | q.s. | q.s. | q.s. |

Procedure:

viii) Methacrylic acid copolymer, Type A and Polycarbophil were passed through sieve #100 and dissolved in a mixture of Isopropyl alcohol /Purified water (2:1).

ix) Iron oxide red was dispersed in small amount of Isopropyl alcohol/Purified water mixture and passed through colloid mill.

x) The contents of step (viii) were mixed with the contents of step (ix) followed by addition of Triethyl citrate in the solution obtained. The solution was stirred for 1-2 hrs.

xi) The granules of step (vii) were coated with the solution of step (x) using Fluidized bed coater (FBC) with either Top spray or Bottom spray technique and dried granules were obtained.

### C. Compression Of Coated Granules

| | | Quantity/tablet (mg) | | | |
|---|---|---|---|---|---|
| S. No. | Ingredients | T-1 | T-2 | T-3 | T-4 |
| 13. | Amoxicillin trihydrate | 604.23 | 684.79 | 1007.04 | 1208.45 |
| | granules coated in FBC | | | | |
| 14. | Microcrystalline | 65.77 | 130.21 | 99.96 | 99.55 |
| | cellulose | | | | |
| | (Avicel ® pH 102) | | | | |
| 15. | Croscarmellose sodium | 40.00 | 50.00 | 50.00 | 50.00 |
| | (Ac-di-sol ®) | | | | |
| 16. | Talc | 5.00 | 10.00 | 10.00 | 10.00 |
| 17. | Magnesium stearate | 5.00 | 10.00 | 10.00 | 10.00 |

Procedure:

xii) Microcrystalline cellulose, Croscarmellose sodium, Talc and Magnesium stearate were blended together and passed through sieve #40.

xiii) The blend obtained in step (xii) was mixed further with a portion of Amoxicillin trihydrate granules.

xiv) The contents of step (xiii) were blended with remaining portion of Amoxicillin trihydrate granules and compressed into tablet.

### D. Coating Of Tablets

| | | Quantity/tablet (mg) | | | |
|---|---|---|---|---|---|
| S. No. | Ingredients | T-1 | T-2 | T-3 | T-4 |
| 18. | Film coating system comprising | 21.00 | 26.00 | 35.00 | 42.00 |
| | Carrageenan and Microcrytalline | | | | |
| | cellulose (Lustreclear ®) | | | | |
| 19. | Purified water** | q.s. | q.s. | q.s. | q.s. |

Procedure:

xv) Film coating system comprising Carrageenan and Microcrytalline cellulose was passed through sieve #60.

xvi) The bulk of step (xv) was dispersed in Purified water followed by stirring the solution for 1-2 hours.

xvii) The tablets of step (xiv) were coated with coating solution of step (xvi).

(‘**’ indicates lost in processing)

### Example—2

### A. Preparation Of Granules

| S. No. | Ingredients | Quantity/tablet (mg) |
|---|---|---|
| 1. | Amoxicillin trihydrate | 862.5 |
| | (Equivalent to Amoxicillin 750 mg) | |
| 2. | Polyethylene oxide | 50.0 |
| 3. | Polycarbophil | 20.0 |
| 4. | Lactose | 30.0 |
| 5. | Croscarmellose sodium | 25.0 |
| 6. | Purified water | Lost in processing |

Procedure:

i) Amoxicillin trihydrate, Polyethylene oxide, Lactose, Croscarmellose sodium and Polycarbophil were passed through sieve #30 followed by mixing.

ii) The blend of step (i) was granulated with Purified water.

iii) The wet mass of step (ii) was passed through sieve #8.

iv) The granules of step (iii) were semi-dried at a temperature of 50° C. and passed through sieve #24 followed by breaking the lumps retained on the sieve.

v) The granules of step (iv) were passed through sieve #80 and further collected.

vi) The undersize granules obtained in step (v) were milled followed by regranulating the granules with purified water. The process of step (iii) was repeated until at least 95% of the material of +#24 and −#80 fraction was obtained.

vii) The total fraction of granules obtained were blended and stored in double polyethylene bags in tightly closed HDPE containers.

### B. Coating Of Granules

| S. No. | Ingredients | Percent (%) w/w |
|---|---|---|
| 7. | Methacrylic acid copolymer, | 20.00 |
| | Type A (Eudragit ® L-100) | |
| 8. | Polycarbophil | 0.30 |
| 9. | Triethyl citrate | 2.00 |
| 10. | Iron oxide red | 0.13 |
| 11. | Isopropyl alcohol | Lost in processing |
| 12. | Purified water | Lost in processing |

Procedure:

viii) Methacrylic acid copolymer, Type A and Polycarbophil were passed through sieve #100 and dissolved in a mixture of Isopropyl alcohol/Purified water (2:1).

ix) Iron oxide red was dispersed in small amount of Isopropyl alcohol/Purified water mixture and passed through colloid mill.

x) The contents of step (viii) were mixed with the contents of step (ix) followed by addition of Triethyl citrate in the solution obtained. The solution was stirred for 1-2 hrs.

xi) The granules of step (vii) were coated with the solution of step (x) using Fluidized bed coater (FBC) with either Top spray or Bottom spray technique and dried granules were obtained.

### C. Compression Of Coated Granules

| S. No. | Ingredients | Quantity/tablet (mg) |
|---|---|---|
| 13. | Amoxicillin trihydrate granules | 1209.00 |
| | coated in FBC | |
| 14. | Microcrystalline cellulose | 99.55 |
| | (Avicel ® pH 102) | |
| 15. | Croscarmellose sodium (Ac-di-sol ®) | 50.00 |
| 16. | Talc | 10.00 |
| 17. | Magnesium stearate | 10.00 |
| 18. | Clavulanate potassium and Microcystalline | 298.00 |
| | cellulose mixture (1:1) | |

Procedure:

xii) Clavulanate potassium and Microcystalline cellulose mixture (1:1), Microcrystalline cellulose, Croscarmellose sodium, Talc and Magnesium stearate were blended together and passed through sieve #40.

xiii) The blend obtained in step (xii) was mixed further with a portion of Amoxicillin trihydrate granules.

xiv) The contents of step (xiii) were blended with remaining portion of Amoxicillin trihydrate granules and compressed into tablet.

### D. Coating Of Tablets

| S. No. | Ingredients | Quantity/tablet (mg) |
|---|---|---|
| 19. | Film coating system comprising | 42.00 |
| | Carrgeenan and Microcrytalline cellulose | |
| | (Lustreclear ®) | |
| 20. | Purified water | Lost in processing |

Procedure:

xv) Film coating system comprising Carrageenan and Microcrytalline cellulose was passed through sieve #60.

xvi) The bulk of step (xv) was dispersed in Purified water followed by stirring the solution for 1-2 hours.

xvii) The tablets of step (xiv) were coated with coating solution of step (xvi).

### Example—3

### A. Preparation Of Granules

| S. No. | Ingredients | Quantity/tablet (mg) |
|---|---|---|
| 1. | Amoxicillin trihydrate | 862.5 |
| | (Equivalent to Amoxicillin 750 mg) | |
| 2. | Polycarbophil (Noveon ® AA1) | 20.0 |
| 3. | Mannitol | 30.0 |
| 4. | Crospovidone | 25.0 |
| 5. | Purified water | Lost inprocessing |

Procedure:

i) Amoxicillin trihydrate, Mannitol, Crospovidone and Polycarbophil were passed through sieve #30 followed by blending of all the above ingredients.

ii) The blend of step (i) was granulated with Purified water. 25 iii) The wet mass of step (ii) was passed through sieve #8.

iv) The granules of step (iii) were semi-dried at a temperature of 50° C. and passed through sieve #24 followed by breaking the lumps retained on the sieve.

v) The granules of step (iv) were passed through sieve #80 and further collected.

vi) The granules obtained in step (v) were milled and passed through sieve #24.

### B. Coating Of Granules

| S. No. | Ingredients | Percent (%) w/w |
|---|---|---|
| 6. | Methacrylic acid copolymer, Type C | 15.00 |
| | (Eudragit ® L-100-55) | |
| 7. | Methyl cellulose | 0.50 |
| 8. | Triethyl Citrate | 1.50 |
| 9. | Yellow ferric oxide | 0.13 |
| 10. | Isopropyl alcohol | Lost in processing |
| 11. | Purified water | Lost in processing |

Procedure:

vii) Methacrylic acid copolymer, Type C and Methyl cellulose were passed through sieve#100 and dissolved in a mixture of Isopropyl alcohol/Purified water (2:1)

viii) Yellow ferric oxide was dispersed in small amount of Isopropyl alcohol/Purified water mixture and passed through colloid mill.

ix) The contents of step (viii) were mixed with the contents of step (vii) followed by addition of Triethyl citrate in the solution obtained. The solution was stirred for 1-2 hrs.

x) The granules of step (vi) were coated with the solution of step (ix) using Fluidized bed coater (FBC) with either Top spray or Bottom spray technique.

### C. Compression Of Coated Granules

| S. | | |
|---|---|---|
| No. | Ingredients | Quantity/tablet (mg) |
| 12. | Amoxicillin trihydrate granules coated | 1156.66 |
| | in FBC | |
| 13. | Dicalcium phosphate | 99.55 |
| 14. | Sodium carboxymethyl cellulose (Solutab ®) | 50.00 |
| 15. | Sodium starch glycollate | 75.00 |
| 16. | Talc | 10.00 |
| 17. | Calcium stearate | 10.00 |

Procedure:

xi) Dicalcium phosphate, Sodium carboxymethyl cellulose, Talc, Sodium starch glycollate and Calcium stearate were blended together and passed through sieve #40.

xii) The material obtained in step (xi) was mixed with Amoxicillin trihydrate granules coated in FBC.

xiii) The material of step (xii) was compressed into tablet.

### Example—4

### A. Preparation Of Granules

| S. No. | Ingredients | Quantity/capsule (mg) |
|---|---|---|
| 1. | Amoxicillin trihydrate | 862.5 |
| | (Equivalent to Amoxicillin 750 mg) | |
| 2. | Methacrylic acid copolymer, | 100.0 |
| | Type A (Eudragit ® L100) | |
| 3. | Calcium sulphate | 30.0 |
| 4. | Sodium starch glycollate | 20.0 |
| 5. | Isopropyl alcohol/Purified water (1:1) | Lost inprocessing |
| 6. | Sodium starch glycollate | 50.00 |
| 7. | Talc | 10.00 |
| 8. | Zinc stearate | 10.00 |

Procedure:

i) Amoxicillin trihydrate, Methacrylic acid copolymer, Calcium sulphate and Sodium starch glycollate were passed through sieve #30 followed by mixing.

ii) The blend of step (i) was granulated with Isopropyl alcohol/Purified water (1:1).

iii) The wet mass of step (ii) was passed through sieve #12.

iv) The granules of step (iii) were dried and passed through sieve #24.

v) Sodium starch glycollate, Talc and Zinc stearate were sifted through sieve #40 and mixed with the material of step (iv).

vi) The material of step (v) was compressed to form minitablets, which were then filled into a gelatin capsule.

### B. Coating Of Capsule

| S. No. | Ingredients | Percent (%) w/w |
|---|---|---|
| 9. | Polyacrylate dispersion 30% | 17.50 |
| | (Eudragit ® NE30D) | |
| 10. | Polyethylene glycol | 1.50 |
| 11. | Talc | 6.25 |
| 12. | Purified water | Lost inprocessing |

Procedure:

vii) Polyacrylate dispersion 30% and Talc passed through sieve #80 and Polyethylene glycol were dispersed in Purified water.

viii) The capsules of step (vi) were coated with the solution of step (vii) and dried.

### Example—5

| S. No. | Ingredients | Quantity/capsule (mg) |
|---|---|---|
| 1. | Amoxicillin trihydrate (Equivalent | 431.25 |
| | to Amoxicillin 375 mg) | |
| 2. | Cloxacillin sodium | 273.00 |
| 3. | Hydroxyethyl cellulose | 63.25 |
| 4. | Xanthan gum | 15.00 |
| 5. | Dextrose | 15.00 |
| 6. | Croscarmellose sodium (Vivasol ®) | 25.50 |
| 7. | Microcrystalline cellulose (RQ ® 102) | 40.00 |
| 8. | Talc | 3.00 |

Procedure:

i) Amoxicillin trihydrate, Cloxacillin sodium, Hydroxyethyl cellulose, Dextrose, Croscarmellose sodium and Xanthan gum were passed through sieve #30 followed by mixing.

ii) The blend of step (i) was roller compacted to form compacts, which were then broken and passed through sieve #30.

iii) Croscarmellose sodium and Microcrystalline cellulose were sifted through sieve #40 and mixed.

iv) The material of step (iii) was added to the material of step (ii) and mixed.

v) The material of step (iv) was filled into a hard gelatin capsule.

### Example—6

| S. No. | Ingredients | Quantity/capsule (mg) |
|---|---|---|
| 1. | Cefaclor monohydrate (Equivalent to | 262.23 |
| | Cefaclor 250 mg) | |
| 2. | Methacrylic acid copolymer, Type C | 115.00 |
| | (Eudragit ® L-100-55) | |
| 3. | Polycarbophil (Noveon AA1) | 25.00 |
| 4. | Lactose | 15.00 |
| 5. | Croscarmellose sodium (Vivasol ®) | 25.50 |
| 6. | Isopropyl alcohol/Purified water (1:1) | Lost in processing |
| 7. | Hydrogenated vegetable oil | 2.20 |
| 8. | Colloidal silicon dioxide | 2.20 |

Procedure:

i) Cefaclor monohydrate, Methacrylic acid copolymer, Lactose, Croscarmellose sodium and Polycarbophil were passed through sieve #30 followed by mixing.

ii) The blend of step (i) was granulated with Isopropyl alcohol/Purified water (1:1).

iii) The wet mass of step (ii) was passed through sieve #12 and dried to obtain granules.

iv) The granules of step (iii) were passed through sieve #30 and collected.

v) Hydrogenated vegetable oil and Colloidal silicon dioxide were sifted through sieve #40 and mixed with the material of step (iv).

vi) The material of step (v) was filled into hard gelatin capsule.

### Example—7

### A. Preparation of Granules

| S. No. | Ingredients | Quantity/tablet (mg) |
|---|---|---|
| 1. | Amoxicillin trihydrate (Equivalent to | 488.75 |
| | Amoxicillin 425 mg) | |
| 2. | Clavulanate potassium/Microcrystalline | 250.00 |
| | cellulose 1:1 mixture (Equivalent to | |
| | 125 mg Clavulanic acid) | |
| 3. | Methacrylic acid copolymer, Type A | 115.00 |
| | (Eudragit ® L-100) | |
| 4. | Polyethylene oxide (Polyox ® WSR 303) | 25.00 |
| 5. | Lactose | 15.00 |
| 6. | Sodium starch glycollate | 25.50 |
| 7. | Purified water | Lost in processing |
| 8. | Isopropyl alcohol | Lost in processing |
| 9. | Magnesium stearate | 7.75 |

Procedure:

i) Amoxicillin trihydrate, Clavulanate potassium/Microcrystalline cellulose 1:1 mixture, Methacrylic acid copolymer, Type A, Lactose, Sodium starch glycollate and Polyethylene oxide were passed through sieve #30 followed by mixing.

ii) The blend of step (i) was granulated with Isopropyl alcohol/Purified water mixture (2:1).

iii) The wet mass of step (ii) was passed through sieve #12 and dried.

i) The granules of step (iii) were passed through sieve #24 and mixed with Magnesium stearate sifted through sieve #40.

v) The material of step (iv) was compressed into tablets.

### B. Coating Of Tablets

| S. No. | Ingredients | Percent (%) w/w |
|---|---|---|
| 10. | Ethyl cellulose Aqueous dispersion | 15.00 |
| 11. | Polycarbophil (Noveon ® AA1) | 0.50 |
| 12. | Polyethylene glycol | 3.00 |
| 13. | Red ferric oxide | 0.12 |
| 14. | Purified water | Lost in processing |

Procedure:

vi) Ethyl cellulose aqueous dispersion and Polycarbophil were passed through sieve #100 and dispersed in Purified water.

vii) Red ferric oxide was dispersed in small amount of Purified water and passed through colloid mill.

viii) The contents of step (vii) were mixed with the contents of step (vi) with stirring to obtain a uniform dispersion.

ix) The tablets of step (v) were coated with the solution of step (viii) and dried.

### Example—8

### A. Preparation Of Amoxicillin Sodium Granules

| S. No. | Ingredients | Quantity/tablet (mg) |
|---|---|---|
| 1. | Amoxicillin sodium (Equivalent to | 530 |
| | Amoxicillin 500 mg) | |
| 2. | Ethyl cellulose | 75 |
| 3. | Sodium alginate | 50 |
| 4. | Dibasic calcium phosphate | 15 |
| 5. | Crospovidone | 30 |
| 6. | Isopropyl alcohol | Lost in processing |

Procedure:

i) Amoxicillin sodium, Ethyl cellulose, Dibasic calcium phosphate, Crospovidone and Sodium alginate were passed through sieve #30 followed by mixing.

ii) The blend of step (i) was granulated with Isopropyl alcohol.

iii) The wet mass of step (ii) was passed through sieve #8 and dried.

iv) The dried granules obtained in step (iii) were milled and passed through sieve #20.

### B. Coating Of Granules

| S. No. | Ingredients | Percent (%) w/w |
|---|---|---|
| 7. | Hydroxypropyl methylcellulose phthalate | 20.00 |
| 8. | Polycarbophil (Noveon ® AA1) | 0.50 |
| 9. | Triethyl citrate | 2.50 |
| 10. | Purified water | Lost in processing |

Procedure:

v) Hydroxypropyl methylcellulose phthalate and Polycarbophil were passed through sieve #100 and dispersed in Purified water followed by the addition of Triethyl citrate with stirring.

vi) The granules of step (iv) were coated with the solution of step (v) followed by drying.

### C. Preparation Of Clavulanate Material

| S. No. | Ingredients | Quantity/tablet (mg) |
|---|---|---|
| 11. | Clavulanate potassium | 125.00 |
| 12. | Microcrystalline cellulose | 125.00 |
| 13. | Low substituted hydroxypropyl cellulose | 6.00 |
| 14. | Magnesium stearate | 1.25 |

Procedure:

vii) Clavulanate potassium, Microcrystalline cellulose and Low substituted hydroxypropyl cellulose were mixed together.

viii) Magnesium stearate was sifted through sieve #40 and added to the material of step (vii) followed by mixing.

### D. Tablet

ix) The blend obtained in step (vi) and the material of step (viii) was compressed into a tablet.

### E. Coating Of Tablets

| S. No. | Ingredients | Quantity/tablet (mg) |
|---|---|---|
| 15. | Polyvinyl pyrrolidone | 50.00 |
| 16. | Purified water | Lost in processing |

Procedure:

x) Polyvinyl pyrrolidone was dissolved in Purified water with stirring.

xi) The bilayer tablets of step (ix) was coated with the material of step (x) and dried.

### Example—9

### A. Preparation Of Granules

| S. No. | Ingredients | Quantity/capsule (mg) |
|---|---|---|
| 1. | Ampicillin trihydrate (Equivalent to | 288.63 |
| | Ampicillin 250 mg) | |
| 2. | Xanthan gum | 25.00 |
| 3. | Methacrylic acid copolymer, Type C | 110.00 |
| | (Eudragit ® L-100-55) | |
| 4. | Lactose | 15.00 |
| 5. | Croscarmellose sodium | 30.00 |
| 6. | Isopropyl alcohol/Purified water (1:1) | Lost in processing |

Procedure:

i) Ampicillin trihydrate, Xanthan gum, Lactose, Croscarmellose sodium and Methacrylic acid copolymer, Type C were passed through sieve #30 followed by mixing.

ii) The blend of step (i) was granulated with Isopropyl alcohol/Purified water (1:1).

iii) The wet mass of step (ii) was passed through sieve #12 and dried.

iv) The granules of step (iii) were passed through sieve #24 and collected.

### B. Coating Of Granules

| S. No. | Ingredients | Percent (%) w/w |
|---|---|---|
| 7. | Ethyl cellulose aqueous dispersion | 15.0 |
| 8. | Polycarbophil (Noveon ® AA1) | 0.50 |
| 9. | Triacetin | 2.50 |
| 10. | Yellow ferric oxide | 0.12 |
| 11. | Purified Water | Lost in processing |

Procedure:

v) Ethyl cellulose aqueous dispersion and Polycarbophil were passed through sieve #100 and dispersed in Purified water.

vi) Yellow ferric oxide was dispersed in small amount of Purified water and passed through colloid mill.

vii) The contents of step (vi) were mixed with the contents of step (v) followed by addition of Triacetin to the solution obtained. The solution was stirred for 1-2 hrs.

viii) The granules of step (iv) were coated with the solution of step (vii) followed by drying.

### C. Preparation Of Clavulanate Material

| S. No. | Ingredients | Quantity/capsule (mg) |
|---|---|---|
| 12. | Clavulanate potassium | 125.00 |
| 13. | Lactose | 125.00 |
| 14. | Croscarmellose sodium | 6.0 |
| 15. | Isopropyl alcohol | Lost in processing |
| 16. | Magnesium stearate | 1.25 |

Procedure:

ix) Clavulanate potassium, Lactose and Croscarmellose sodium were sifted through sieve #40 and mixed together.

x) The material of step (ix) was granulated with Isopropyl alcohol followed by drying and sifting of granules through sieve #24.

xi) Magnesium stearate was sifted through sieve #40 and added to the material of step (x) followed by mixing.

### D. Capsule

xii) The material obtained in step (viii) and the material of step (xi) were mixed in 1:1 ratio and filled into hard gelatin capsule.

### Example—10

| S. No. | Ingredients | Quantity/capsule (mg) |
|---|---|---|
| 1. | Ofloxacin | 200.0 |
| 2. | Methacrylic acid copolymer, Type C | 45.0 |
| | (Eudragit ® L-100-55) | |
| 3. | Microcrystalline cellulose | 45.0 |
| 4. | Lactose | 15.0 |
| 5. | Croscarmellose sodium (Solutab ®) | 30.0 |
| 6. | Glyceryl behenate | 2.0 |

Procedure:

i) Ofloxacin, Methacrylic acid copolymer, Type C, Lactose, Microcrystalline cellulose and Croscarmellose sodium were passed through sieve #30 followed by mixing.

ii) Glyceryl behenate was sifted through sieve #40 and mixed with the material of step (i).

iii) The material of step (ii) was filled into capsule.

### Example—11

### A. Preparation Of Granules

| S. No. | Ingredients | Quantity/tablet (mg) |
|---|---|---|
| 1. | Amoxicillin trihydrate (Equivalent to | 720.0 |
| | Amoxicillin 625 mg) | |
| 2. | Polycarbophil (Noveon ® AA1) | 145.0 |
| 3. | Hydroxypropylmethyl cellulose E-15 | 15.0 |
| 4. | Ponceau 4R Supra | 4.0 |
| 5. | Microcrystalline cellulose RQ 102 | 160.0 |
| 6. | Sodium starch glycollate | 25.0 |
| 7. | Croscarmellose sodium (Ac-di-sol ®) | 15.0 |
| 8. | Glyceryl behenate (Compritol ® ATO 88) | 50.0 |
| 9. | Isopropyl alcohol/Dichloromethane | Lost iprocessing |

Procedure:

i) Amoxicillin trihydrate and Polycarbophil were blended together.

ii) Hydroxypropylmethyl cellulose E-15 was dissolved in 1:2 mixtures of Isopropyl alcohol/Dichloromethane.

iii) Ponceau 4R Supra was passed through sieve #100 and was blended with the contents of step (i).

iv) The blend obtained in step (iii) was granulated with the contents of step (ii) and the wet mass was passed through sieve # 15.

v) The wet mass obtained in step (iv) was dried and passed through sieve#24.

vi) Microcrystalline cellulose, Sodium starch glycollate, Croscarmellose sodium, Glyceryl behenate were blended together with the dried mass of step (v) and compressed into tablets.

### B. Coating Of Tablets:

| S. No. | Ingredients | Percent (%) w/w |
|---|---|---|
| 10. | Aminoalkyl methacrylate copolymer E | 10.0 |
| | (Eudragit ® EPO) | |
| 11. | Talc | 0.6 |
| 12. | Polyethylene glycol 400 | 10.0 |
| 13. | Isopropyl alcohol | 100.0 |
| 14. | Dichloromethane | 300.0 |

Procedure:

vii) Aminoalkyl methacrylate copolymer E and Polyethylene glycol 400 were dissolved in a mixture of Isopropyl alcohol and Dichloromethane and stirred for 30-60 mins,

viii) Talc was passed through sieve#200 and dispersed in solution of step (vii).

ix) The tablets of step (vi) were coated with the solution obtained in step (viii).

### Example—12

### A. Fast Release Fraction:

| S. No. | Ingredient | Quantity/capsule (mg) |
|---|---|---|
| 1. | Amoxicillin trihydrate (Equivalent to | 435.0 |
| | Amoxicillin 375 mg) | |
| 2. | Lactose | 40.0 |
| 3. | Sodium starch glycollate | 15.0 |
| 4. | Colloidal silicon dioxide | 12 |
| 5. | Povidone K-30 | 20 |
| 6. | Starch | 5.0 |
| 7. | Polysorbate 80 | 1.0 |
| 8. | Purified water | Lost in processing |
| 9. | Magnesium stearate | 10 |
| 10. | Croscarmellose sodium | 8.0 |

Procedure:

i) Amoxicillin trihydrate, Lactose, Sodium starch glycollate, Colloidal silicon dioxide were mixed together and sifted through mesh #30 sieve

ii) Povidone K-30, Starch, Polysorbate 80 was dissolved together in Purified water to form a homogeneous solution.

iii) The material of step (i) was mixed with the material of step (ii) followed by drying and sifting through mesh #16 sieve.

iv) Magnesium stearate and Croscarmellose sodium were sifted through mesh #40 sieve.

v) The material of step (iv) was mixed with the material of step (iii).

### B. Sustained Release Fraction

| S. No. | Ingredient | Quantity/capsule (mg) |
|---|---|---|
| 11. | Amoxicillin trihydrate (Equivalent to | 435.0 |
| | Amoxicillin 375 mg) | |
| 12. | Lactose monohydrate | 40.0 |
| 13. | Methacrylic Acid Copolymer, Type A | 60.0 |
| | (Eudragit ® L-100) | |
| 14. | Docusate sodium | 5.0 |
| 15. | Hydroxypropyl methylcellulose | 12.0 |
| 16. | Purified water | Lost in processing |
| 17. | Colloidal silicon dioxide | 10.0 |
| 18. | Magnesium stearate | 8.0 |

Procedure

vi) Amoxicillin trihydrate, Lactose monohydrate, Methacrylic Acid Copolymer, Type A were mixed together and sifted through mesh #30 sieve.

vii) Docusate sodium, Hydroxypropyl methylcellulose were dissolved in Purified water to obtain a homogeneous dispersion.

viii) The material of step (vi) was granulated with the material of step (vii) followed by drying and sifting through mesh #24 sieve.

ix) Colloidal silicon dioxide and Magnesium stearate were sifted through mesh #40 sieve.

x) The material of step (ix) was mixed with the material of step (viii).

### C. Capsule

xi) The material obtained in step (v) and the material obtained in step (x) were mixed together and filled into hard gelatin capsule

1. A modified release pharmaceutical dosage form composition which comprises at least one antibiotic(s) or a pharmaceutically acceptable salts, esters, polymorphs, isomers, prodrugs, solvates, hydrates, or derivatives thereof as an active ingredient treated with at least one release modifying agent optionally with one or more other pharmaceutically acceptable excipient(s), wherein the dosage form provides a release of not more than about 60% of the antibiotic in about 30 minutes and not less than about 70% of the antibiotic after 8 hours when subjected to in vitro dissolution study or when tested in vivo.

2. The modified release pharmaceutical dosage form composition according to claim 1, wherein the dosage form composition provides an in vitro release of not more than about 60% of beta-lactam antibiotic in 30 minutes and not less than about 70% of the beta-lactam antibiotic after 8 hours when tested by the USP Apparatus Type II at 75 rpm, 37±0.5° C. and using 900 ml of distilled water as dissolution media, or equivalent conditions.

3. The modified release pharmaceutical dosage form composition according to claim 1, which when tested in a group of healthy humans provides a mean peak plasma concentration (Cₘₐₓ) after at least about 0.5 hour of administration of the dosage form.

4. The modified release pharmaceutical dosage form composition according to claim 1, which when tested in a group of healthy humans provides a mean peak plasma concentration (Cₘₐₓ) within 0.5-12 hours.

5. The modified release pharmaceutical dosage form composition according to claim 1, which when tested in humans showed a mean peak plasma concentration (Cₘₐₓ) of amoxicillin in the range of about 0. 1-50 mg/ml.

6. The modified release pharmaceutical dosage form composition according to claim 1, wherein the dosage form composition provides a in vitro release of not more than about 60% of the beta-lactam antibiotic in about 30 minutes and not less than about 70% of the beta-lactam antibiotic after about 8 hours as tested by the USP Apparatus Type II at 75 rpm, 37±0.5° C. and using 900 ml of Distilled water or 0.01N HCl as dissolution media, and when tested in a group of healthy humans the mean peak plasma concentration (Cₘₐₓ) is achieved after at least about 0.5 hour of administration of the dosage form.

7. The modified release pharmaceutical dosage form composition according to claim 1, which provides a release of not less than about 80% of the antibiotic after about 8 hours of dissolution study conducted using 900 ml of pH 7.4 Phosphate buffer in USP Apparatus Type II (paddles method) at 75 rpm.

8. The modified release pharmaceutical dosage form composition according to claim 1, which provides a release of about 0-50% of the antibiotic within about 2 hours and greater than about 40% of the active ingredient(s) after about 8 hours of test when subjected to in vitro dissolution study in dissolution media having a pH ranging from about 1 to about 5.5.

9. The modified release pharmaceutical dosage form composition according to claim 1, wherein the antibiotic active ingredient is selected from a group consisting of amoxicillin, ampicillin, bacampicillin, carbenicillin, cloxacillin, dicloxacillin, flucloxacillin, methicillin, mezlocillin, nafcillin, oxacillin, penicillin G, penicillin V, piperacillin, pivampicillin, pivmecillinam, ticarcillin, clavulanic acid; ciprofloxacin, ofloxacin, and levofloxacin, and a pharmaceutically acceptable salt, ester, polymorph, isomer, prodrug, solvate, hydrate, or derivative thereof and mixtures thereof.

10. The modified release pharmaceutical dosage form composition according to claim 1, wherein the antibiotic is amoxicillin or a pharmaceutically acceptable salts, esters, polymorphs, isomers, prodrugs, solvates, hydrates, or derivatives thereof.

11. The modified release pharmaceutical dosage form composition according to claim 1, which is designed for once-a-day or twice-a-day administration and which releases the amoxicillin in a desired manner particularly in vivo so as to maintain therapeutic levels of the drug in the plasma for extended periods of time devoid of or at least minimized adverse effects associated with antibiotic therapy.

12. The modified release pharmaceutical dosage form composition according to claim 11, wherein the dosage form composition provides an in vitro dissolution of not less than about 5% and not more than about 60% of the antibiotic release after 0.5 hours; from not less than about 15% amoxicillin is released in 3 hours; and not less than about 60% amoxicillin released in 6 hours as tested by the USP Apparatus Type II at 75 rpm, 37±0.5° C. and 900 ml of Distilled water as the dissolution medium.

13. The modified release pharmaceutical dosage form composition according to claim 1, comprising at least two antibiotics as active ingredients.

14. The modified release pharmaceutical dosage form composition according to claim 13, wherein the antibiotic active ingredients are amoxicillin trihydrate and clavulanate potassium.

15. The modified release pharmaceutical dosage form composition according to claim 14, which comprises amoxicillin trihydrate equivalent to about 300 to about 1650 mg of amoxicillin and clavulanate potassium equivalent to about 62.5 to about 300 mg of clavulanic acid with at least one release modifying agent(s) optionally with one or more other pharmaceutically acceptable excipient(s).

16. The A modified release pharmaceutical dosage form composition according to claim 14, which comprises amoxicillin trihydrate equivalent to about 425 mg to about 1500 mg of amoxicillin, and clavulanate potassium equivalent to about 125 mg to about 250 mg of clavulanic acid with at least one release modifying agent(s) optionally with one or more other pharmaceutically acceptable excipient(s).

17. The modified release pharmaceutical dosage form composition according to claim 14, which comprises amoxicillin formulated with at least one release modifying agent(s) and one or more other pharmaceutically acceptable excipient(s) to provide an extended release of amoxicillin, and potassium clavulanate formulated with one or more pharmaceutically acceptable excipient(s) in an immediate release form to provide immediate or fast release of clavulanate.

18. The modified release pharmaceutical dosage form composition according to claim 14, wherein the potassium clavulanate provides a release of not less than about 20% of the antibiotic in about 2 hours and about 75% in about 1 to about 15 hours when subjected to in vitro test using USP Apparatus Type II at 75 rpm, 37±0.5° C. and using 900 ml of distilled water or 0.0 IN HCl as dissolution media.

19. The modified release pharmaceutical dosage form composition according to claim 1, wherein the time over MIC (T>MIC) for the antibiotic compositions is at least 40% at a concentration of at least about 0.25 μg/ml of the antibiotic at the said MIC.

20. The modified release pharmaceutical dosage form composition according to claim 1, wherein the antibiotic compositions provide therapeutic levels of the antibiotic active ingredient at concentrations of about 0.25 μg/ml of the antibiotic for at least about 4-6 hours after administration or for such time as required to provide effectiveness of the antibiotic.

21. The modified release pharmaceutical dosage form composition according to claim 1, wherein the compositions reduces the adverse effects or side effects associated with the antibiotic(s) by controlling the peak plasma concentration (Cₘₐₓ) such that the concentration of the antibiotic(s) is substantially below its toxic levels at any point of time although the plasma concentration of the antibiotic(s) is above the MIC for such period adequate to provide the therapeutic efficacy.

22. The modified release pharmaceutical dosage form composition according to claim 1, wherein the release controlling agent is a mucoadhesive polymer selected from polycarbophil and polyethylene oxide and a mixture thereof which reduces the side effects particularly in the form of gastrointestinal disorders/disturbances related to the antibiotic(s) therapy.

23. The modified release pharmaceutical dosage form composition according to claim 1, wherein the composition has such a Cₘₐₓ to MIC ratio so as to avoid or at least minimize development of resistant microbial strains.

24. The modified release pharmaceutical dosage form composition according to claim 23, wherein the Cₘₐₓ value is at least two to three times the MIC value.

25. The modified release pharmaceutical dosage form composition according to claim 1, wherein the composition comprises a plurality of particles, wherein each particle comprises at least one antibiotic(s) or a pharmaceutically acceptable salts, esters, polymorphs, isomers, prodrugs, solvates, hydrates, or derivatives thereof, treated with at least one release modifying agent optionally with one or more pharmaceutically acceptable excipient(s) for controlling the release of the antibiotic(s).

26. The modified release pharmaceutical dosage form composition according to claim 1, wherein the release modifying agent is selected from the group comprising carbopol; cellulosic polymers; copolymers of methyl vinyl ether and maleic anhydride; enteric polymers; sodium hyaluronate; gums; alginates; polycarbophil; polyethylene oxide; starch; dextran; chitosan; and a mixture thereof.

27. The modified release pharmaceutical dosage form composition according to claim 1, wherein the release modifying agent comprises a polymeric material selected from the group consisting of pH dependent polymers; pH independent polymers; swellable polymers; non-swellable polymers; hydrophilic polymers; hydrophobic polymers one or more other hydrophobic materials; ionic polymers; non-ionic polymers; synthetic or natural polysaccharides, and a mixture thereof.

28. The modified release pharmaceutical dosage form composition according to claim 1, wherein the dosage form additionally comprises at least one surfactant selected from a group comprising anionic surfactants, cationic surfactants, non-ionic surfactants, zwitterionic surfactants and a mixture or mixtures thereof.

29. The modified release pharmaceutical dosage form composition according to claim 1, wherein the other pharmaceutically acceptable excipients are selected from a group comprising diluents; disintegrants; binders; fillers; bulking agent; vehicles, organic acid(s); colorants; stabilizers; preservatives; lubricants; glidants; chelating agents; vehicles; bulking agents; stabilizers; preservatives; hydrophilic polymers; solubility enhancing agents; tonicity adjusting agents; local anesthetics; pH adjusting agents; antioxidants; osmotic agents; chelating agents; viscosifying agents; acids; sugar alcohol; reducing sugars; and non-reducing sugars used either alone or in combination thereof.

30. A process of preparation of a modified release pharmaceutical dosage form composition according to claim 1, which comprises treating the antibiotic(s) or a pharmaceutically acceptable salts, esters, polymorphs, isomers, prodrugs, solvates, hydrates, or derivatives thereof, with at least one release modifying agent(s) optionally with one or more other pharmaceutically acceptable excipient(s) and formulating it into the desired dosage form.

31. A method for prophylaxis, amelioration and/or treatment of a bacterial infection using the modified release pharmaceutical dosage form composition according to claim 1, which comprises administering to a subject in need thereof an effective amount of the composition.

32. The method according to claim 31, wherein the bacterial infection is an upper respiratory tract infections.

33. (canceled)

34. (canceled)

35. The method according to claim 32, wherein the upper respiratory tract infection is manifested as a sore throat, acute bacterial tonsillitis, pharyngitis or a combination thereof.

36. The method according to claim 31, wherein the bacterial infection is due to an infection by one or more microorganisms selected from gram positive and gram negative bacteria.

37. The modified release pharmaceutical dosage form composition according to claim 2, which when tested in a group of healthy humans provides a mean peak plasma concentration (Cₘₐₓ) after at least about 0.5 hour of administration of the dosage form.

38. The modified release pharmaceutical dosage form composition according to claim 2, which when tested in a group of healthy humans provides a mean peak plasma concentration (Cₘₐₓ) within 0.5-12 hours.

39. The modified release pharmaceutical dosage form composition according to claim 2, which when tested in humans showed a mean peak plasma concentration (Cₘₐₓ) of amoxicillin in the range of about 0.1-50 μg/ml.

40. The modified release pharmaceutical dosage form composition according to claim 3, which when tested in humans showed a mean peak plasma concentration (Cₘₐₓ) of amoxicillin in the range of about 0.1-50 μg/ml.

41. The modified release pharmaceutical dosage form composition according to claim 4, which when tested in humans showed a mean peak plasma concentration (Cₘₐₓ) of amoxicillin in the range of about 0.1-50 μg/ml.

42. The modified release pharmaceutical dosage form composition according to claim 37, which when tested in humans showed a mean peak plasma concentration (Cₘₐₓ) of amoxicillin in the range of about 0.1-50 μg/ml.

43. The modified release pharmaceutical dosage form composition according to claim 38, which when tested in humans showed a mean peak plasma concentration (Cₘₐₓ) of amoxicillin in the range of about 0.1-50 μg/ml.

44. The modified release pharmaceutical dosage form composition according to claim 2, wherein the antibiotic active ingredient is selected from a group comprising of amoxicillin, ampicillin, bacampicillin, carbenicillin, cloxacillin, dicloxacillin, flucloxacillin, methicillin, mezlocillin, nafcillin, oxacillin, penicillin G, penicillin V, piperacillin, pivampicillin, pivmecillinam, ticarcillin, clavulanic acid; ciprofloxacin, ofloxacin, and levofloxacin, and a pharmaceutically acceptable salt, ester, polymorph, isomer, prodrug, solvate, hydrate, or derivative thereof and mixtures thereof.
